(19) United States
(12) Patent Application Publication
(10) Pub. No.: US 2015/0320402 A1
(43) Pub. Date: Nov. 12, 2015

(54) ULTRASOUND PROBE CAP AND METHOD FOR TESTING ULTRASOUND PROBE USING THE SAME AND ULTRASOUND DIAGNOSIS SYSTEM THEREOF

(71) Applicant: HEALCERION CO., LTD., Seoul (KR)

(72) Inventors: Jeong Won RYU, Seoul (KR); You Chan CHOUNG, Seoul (KR)

(21) Appl. No.: 14/647,246

(22) PCT Filed: Dec. 12, 2013
(86) PCT No.: PCT/KR2013/011512

§ 371 Date: May 26, 2015

(30) Foreign Application Priority Data
Dec. 14, 2012 (KR) 10-2012-0146453

(51) Int. Cl. A61B 8/00; A61B 8/14

(52) U.S. Cl. CPC A61B 8/58; A61B 8/14; A61B 8/4444

(57) ABSTRACT

Provided is an ultrasound diagnosis system including an ultrasound probe apparatus transmitting an ultrasound signal to obtain an ultrasound image of a subject, and an ultrasound diagnosis apparatus constructing a database for ultrasound echo data of a normal operation of the piezoelectric device module provided in the ultrasound probe apparatus.

### TECHNICAL FIELD

The present invention relates to an ultrasound diagnosis system, and more particularly, to an ultrasound probe protection cap capable of protecting an ultrasound probe while keeping the same and individually checking an operation state and function of ultrasound piezoelectric devices multiply arranged in an array and an ultrasound probe test method and an ultrasound diagnosis system, using the cap.

### BACKGROUND ART

Since having noninvasive and nondestructive properties, ultrasound diagnosis systems are generally used in the medical field to obtain information of the inside of an object. Since high-resolution images of internal organizations of objects may be provided to doctors with no surgical operations of directly incising and observing objects, ultrasound diagnosis systems are very importantly used in the medical field.

Ultrasound diagnosis systems are a system that emits an ultrasound signal from a body surface of a subject toward a target portion inside a body of the subject, extracts information from a reflected ultrasound signal, and obtains an image of a section of soft tissue or a blood flow in a noninvasive manner.

Comparing with other imaging diagnosis apparatuses such as X-ray inspection apparatuses, computerized tomography (CT) scanners, magnetic resonance image (MRI) scanners, and nuclear medicine inspection apparatuses, ultrasound diagnosis systems described above have a small size, are cheap, may display in real time, and have excellent safety without being exposed to X-rays, thereby being generally used to diagnose hearts, internal organs in an abdominal cavity, urinary systems, and genital organs.

Ultrasound diagnosis systems each includes a probe for transmitting an ultrasound signal to a subject in order to obtain an ultrasound image of the subject and receiving the ultrasound signal reflected from the subject.

Since ultrasound probes employ piezoelectric devices very vulnerable to a shock as primary components, it is necessary to give heed to ultrasound probes while ultrasound probes are being stored after use. It is impossible to repair piezoelectric devices once damaged and it is necessary to completely replace damaged piezoelectric devices. Also, it may not be recognized before use whether piezoelectric devices are damaged or not.

### DISCLOSURE OF INVENTION

### Technical Problem

The present invention provides an ultrasound probe protection cap capable of protecting an ultrasound probe while keeping the same and individually checking an operation state and function of ultrasound piezoelectric devices multiply arranged in an array and an ultrasound probe test method and an ultrasound diagnosis system, using the cap.

### Solution to Problem

According to an aspect of the present invention, there is provided an ultrasound diagnosis system including an ultrasound probe apparatus transmitting an ultrasound signal to obtain an ultrasound image of a subject, receiving the ultrasound signal reflected from the subject, emitting ultrasound to a probe cap to test a provided piezoelectric device module, and receiving an ultrasound echo obtained from the probe cap and an ultrasound diagnosis apparatus constructing a database for ultrasound echo data of a normal operation of the piezoelectric device module provided in the ultrasound probe apparatus, executing a probe test mode for testing an operation of the piezoelectric device module, receiving data of the ultrasound echo obtained from the probe cap from the ultrasound probe apparatus, comparing with the ultrasound echo data of the normal operation of the piezoelectric device module in the database, and displaying whether the piezoelectric device module is normal or defective as a test result.

The ultrasound probe apparatus may include a probe body transmitting the ultrasound signals to obtain the ultrasound image of the subject and to perform the probe test and receiving and transmitting the reflected ultrasound signals to the ultrasound diagnosis apparatus and a probe cap coupled with a front end of the probe body, protecting the probe body and generating the ultrasound echo while testing the piezoelectric device module of the probe body.

The probe cap may include a housing forming an external shape of the probe cap and protecting the probe body from a damage caused by an external force while being coupled with the probe body, an inside thereof formed of a material having an ultrasound reflection coefficient to allow the probe test and a buffer formed on an inner surface of the housing to be allowed to be in surface contact with the front end of the probe body while the probe cap is being coupled with the probe body and formed of a buffering material having an ultrasound reflection coefficient corresponding to acoustic impedance inside a human body to allow the probe test.

According to another aspect of the present invention, there is provided a probe test method of an ultrasound diagnosis system including an ultrasound probe apparatus and an ultrasound diagnosis apparatus. The method includes, the ultrasound diagnosis apparatus, constructing a database for ultrasound echo data of a normal operation of a piezoelectric device module provided in the ultrasound probe apparatus, the ultrasound diagnosis apparatus, executing a probe test mode for testing an operation of the piezoelectric device module, the ultrasound diagnosis apparatus, when the ultrasound probe apparatus emits ultrasound to a probe cap in the ultrasound probe apparatus to test the piezoelectric device module provided in the corresponding ultrasound probe apparatus, receiving ultrasound echo data obtained from the probe cap from the ultrasound probe apparatus, and the ultrasound diagnosis apparatus, comparing the ultrasound echo data obtained from the probe cap, received from the ultrasound probe apparatus, with the ultrasound echo data of the normal operation of the piezoelectric device module in the database and displaying whether the piezoelectric device module is normal or defective as a test result.

According to still another aspect of the present invention, there is provided an ultrasound probe apparatus forming an ultrasound diagnosis system together with an ultrasound diagnosis apparatus. The ultrasound probe apparatus includes a probe body transmitting an ultrasound signal to obtain an ultrasound image of a subject to the subject, receiving the ultrasound signal reflected from the subject, emitting ultrasound to test a provided piezoelectric device module, and receiving an ultrasound echo reflected therefrom and a probe cap coupled with a front end of the probe body, protecting the probe body and generating the ultrasound echo while testing the piezoelectric device module of the probe body.

The probe cap may include a housing forming an external shape of the probe cap and protecting the probe body from a damage caused by an external force while being coupled with the probe body, an inside thereof formed of a material having an ultrasound reflection coefficient to allow the probe test and a buffer formed on an inner surface of the housing to be allowed to be in surface contact with the front end of the probe body while the probe cap is being coupled with the probe body and formed of a buffering material having an ultrasound reflection coefficient corresponding to acoustic impedance inside a human body to allow the probe test.

According to yet another aspect of the present invention, there is provided a probe test method of an ultrasound probe apparatus forming an ultrasound diagnosis system together with an ultrasound diagnosis apparatus. The method may include, the ultrasound probe apparatus, constructing a database for ultrasound echo data of a normal operation of a piezoelectric device module provided in the corresponding ultrasound probe apparatus, the ultrasound probe apparatus, executing a probe test mode for testing an operation of the piezoelectric device module, the ultrasound probe apparatus emitting ultrasound to a probe cap in the corresponding ultrasound probe apparatus to test the piezoelectric device module provided in the ultrasound probe apparatus and receiving ultrasound echo data obtained from the probe cap, and the ultrasound probe apparatus, comparing the received ultrasound echo data obtained from the probe cap with the ultrasound echo data of the normal operation of the piezoelectric device module in the database and displaying whether the piezoelectric device module is normal or defective as a test result.

### Advantageous Effects of Invention

According to the embodiments of the present invention, a probe cap is provided in an ultrasound probe apparatus, thereby protecting a piezoelectric device module in the ultrasound probe apparatus from external contacts or shocks.

Also, according to the embodiments of the present invention, in a probe test mode, operation states and functions of ultrasound piezoelectric devices multiply arranged in an array are individually checked and visually displayed while the probe cap of the ultrasound probe apparatus is coupled with a probe body, thereby preventing an error in ultrasound diagnosis and stably managing the ultrasound probe apparatus by providing a user with present state information of the ultrasound probe apparatus.

### BRIEF DESCRIPTION OF DRAWINGS

FIG. 1 is a block view illustrating an ultrasound diagnosis system according to an embodiment of the present invention;

FIG. 2 is a view illustrating various probe bodies according to an embodiment of the present invention;

FIGS. 3 and 4 are views illustrating an ultrasound probe apparatus according to an embodiment of the present invention;

FIG. 5 is a view illustrating a piezoelectric device module according to an embodiment of the present invention;

FIG. 6 is a view illustrating an echo phenomenon of ultrasound according to an embodiment of the present invention;

FIG. 7 is a flowchart illustrating a probe test method of an ultrasound diagnosis system of FIG. 1 according to an embodiment of the present invention;

FIG. 8 is a block view illustrating an ultrasound diagnosis system according to another embodiment of the present invention; and

FIG. 9 is a flowchart illustrating a probe test method of the ultrasound diagnosis system of FIG. 8 according to another embodiment of the present invention.

### BEST MODE FOR CARRYING OUT THE INVENTION

Hereinafter, embodiments of the present invention will be described in detail with reference to the attached drawings. The following embodiments are provided as examples to fully convey the inventive concept to those skilled in the art. Accordingly, the present invention is not limited to the embodiments described below and may be embodied in other forms. Also, in the drawings, a width, a length, and a thickness of an element may be exaggerated for convenience of description. Like reference numerals designate like elements throughout.

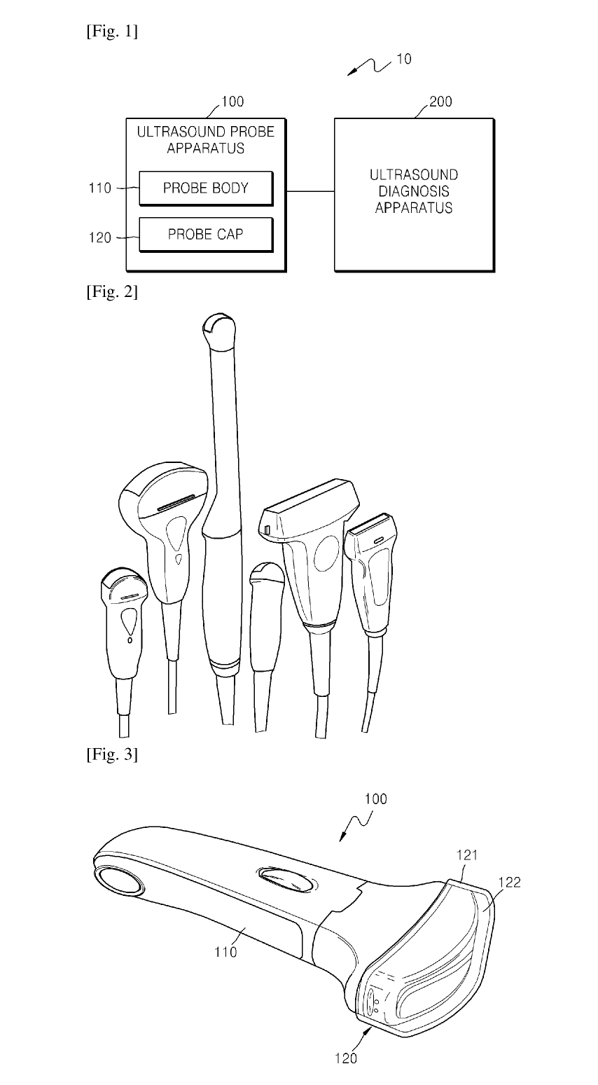

FIG. 1 is a block view illustrating an ultrasound diagnosis system 10 according to an embodiment of the present invention.

Referring to FIG. 1, the ultrasound diagnosis system 10 may include an ultrasound probe apparatus 100 and an ultrasound diagnosis apparatus 200.

The ultrasound probe apparatus 100 may include a probe body 110 and a probe cap 120. The probe cap 120 is coupled with a front end of the probe body 110 and protects the probe body 110. In addition, the probe cap 120 may generate an ultrasound echo while testing piezoelectric devices of the probe body 110.

The probe body 110, as shown in FIG. 2, may be embodied as various shapes. The probe body 110 transmits an ultrasound signal to a subject in order to obtain an ultrasound image of the subject and receives and transmits the ultrasound signal reflected from the subject to the ultrasound diagnosis apparatus 200. In addition, when the ultrasound diagnosis apparatus 200 executes a probe test mode while the probe cap 120 is being coupled with the probe body 110, the probe body 110 generates ultrasound for a probe test and transmits an ultrasound echo reflected from the probe cap 120 to the ultrasound diagnosis apparatus 200.

For this, the probe body 110 may include an ultrasound probe, a beam former, a data processor, a scan converter, and a display unit. The ultrasound probe may transmit an ultrasound signal to an object and may receive the ultrasound signal reflected from the object, that is, an ultrasound echo signal, thereby forming a reception signal. The ultrasound probe may include at least one transducer element operating to convert an ultrasound signal and an electric signal into each other. The beam former may analog/digital-convert the reception signal provided by the ultrasound probe, may delay a time of a digital signal considering a position and a focusing point of each transducer element, and forms ultrasound data, that is, radio frequency (RF) data by summing up the time-delayed digital signals. The data processor performs various data processes with respect to ultrasound data, which are necessary for forming an ultrasound image. The scan converter scan-converts the processed ultrasound data to be displayed as an image. The display unit may display an operation state of the probe body 110.

The ultrasound diagnosis apparatus 200 may receive the ultrasound data from the ultrasound probe apparatus 100 and may provide an ultrasound image having high-resolution with respect to internal organs of an object for ultrasound diagnosis with no surgical operation of directly incising and observing the object.

The ultrasound diagnosis apparatus 200 may include various devices having a wireless communication function and a display unit and operating application programs. For example, there may be a personal computer, a smartphone, a tablet type device, a pad type device, and a personal digital assistance (PDA).

Since ultrasound is absorbed into a human body due to properties thereof, the ultrasound reflected by a deep part and returning late has a great loss of energy and is reduced in size. In the same human anatomy, a size of ultrasound data reflected by a deeper part has a relatively smaller size. Accordingly, it is necessary to compensate a value increased in proportion to an amount of time for being reflected and returning.

The ultrasound diagnosis apparatus 200 may generate an ultrasound image of ultrasound data compensated with a time gain and adjusted with brightness and contrast with respect to the ultrasound data received from the ultrasound probe apparatus 100 and may display the ultrasound image on the display unit.

The ultrasound diagnosis apparatus 200 may determine an ultrasound measurement depth according to an input of a user, may determine a parameter for adjusting the time gain based on the ultrasound measurement depth, and may determine degrees for adjusting brightness and contrast.

The ultrasound diagnosis apparatus 200 may receive an input of the user and may transmit the input to the ultrasound probe apparatus 100. For example, the ultrasound diagnosis apparatus 200 may transmit the determined ultrasound measurement depth to the ultrasound probe apparatus 100 to control the ultrasound probe apparatus 100.

Also, the ultrasound diagnosis apparatus 200 may execute a probe test mode for testing an operation of a piezoelectric device module of the ultrasound probe apparatus 100 and may display whether the piezoelectric device module is normal or defective, as a test result.

For this, the ultrasound diagnosis apparatus 200 may construct a database for an ultrasound echo of a normal operation of the piezoelectric device module provided in the ultrasound probe apparatus 100.

Herein, the database for the ultrasound echo of the normal operation of the piezoelectric device module generates ultrasound having a certain frequency by applying an electric pulse having a certain frequency to the piezoelectric device module and is constructed based on an ultrasound echo having a certain frequency generated by the generated ultrasound having the certain frequency.

That is, herein, a meaning of “the normal operation” is a normal operation based on data of the time when ultrasound having a certain frequency is applied to skin tissues by applying an electric pulse to the piezoelectric device module and echo data is obtained based on data of the ultrasound having the certain frequency obtained when the ultrasound is reflected by an interface of the skin tissues.

The ultrasound diagnosis apparatus 200 may execute a probe test mode for testing an operation of the piezoelectric device module to receive ultrasound echo data obtained by the probe cap 120 from the ultrasound probe apparatus 100 to compare with the database for the ultrasound echo data of the normal operation of the piezoelectric device module, thereby displaying whether the piezoelectric device module is normal or defective, as a test result.

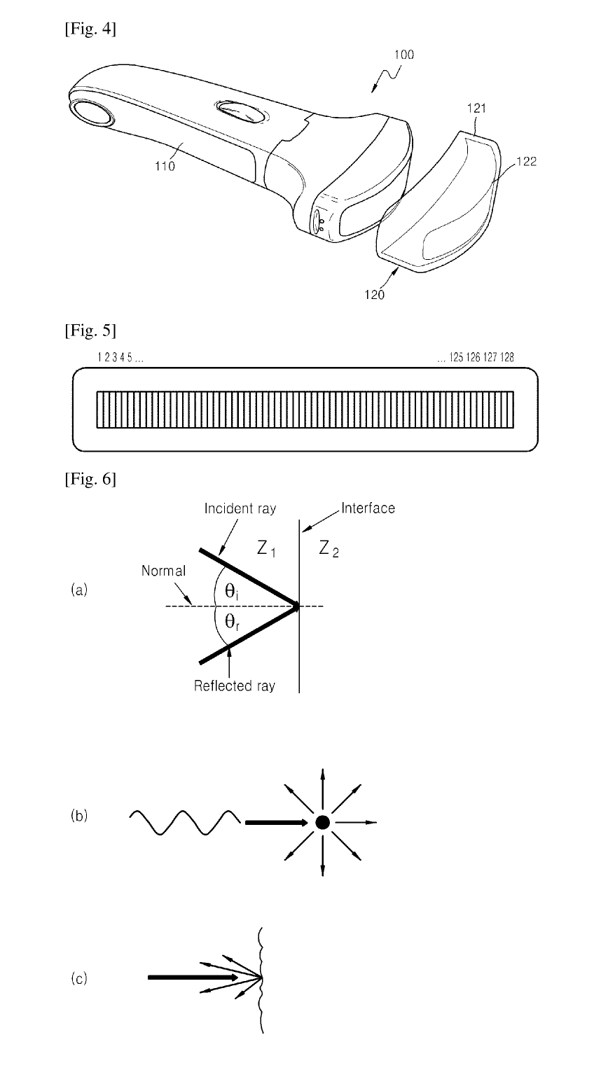

FIGS. 3 and 4 are views illustrating the ultrasound probe apparatus 100.

The ultrasound probe apparatus 100 may include the probe body 110 and the probe cap 120.

The probe body 110 includes a piezoelectric device module built inside a front thereof. Piezoelectric devices are formed of a piezoelectric material. The piezoelectric material may oscillate to generate and transmit pulses of a sound wave into a human body and may receive and convert a reflected echo into an electric signal. Recently, as the piezoelectric material, piezoelectric ceramic such as lead zirconatetitanate (PZT) having highest acoustoelectric conversion efficiency is generally used. The piezoelectric device module may be used while a large number such as 64, 128, and 192 of piezoelectric devices are being arranged in an array.

FIG. 5 is a view illustrating the piezoelectric device module according to an embodiment of the present invention.

Referring to FIG. 5, the piezoelectric device module may be formed of 128 of individual piezoelectric devices. The piezoelectric device module may include ultrasound piezoelectric devices multiply arranged in an array.

Accordingly, in order to determine whether the individual piezoelectric devices forming the piezoelectric device module is normally operating or not, a test capable of individually checking an operation state and function thereof is necessary.

The ultrasound diagnosis apparatus 200 may perform tests for the individual piezoelectric devices one by one by using the probe cap 120.

When sending ultrasound outputted by the probe body 110 into a human body, at an interface between soft tissues having different densities, the ultrasound is reflected, thereby generating an ultrasound echo. The reflected ultrasound echo returns to the probe body 110. The ultrasound diagnosis 200 analyzes and processes a difference in strength of the echo received from the probe body 110 to be displayed with brightness of dots, thereby forming an image.

The probe cap 120 may be coupled with or separated from the probe body 110 through a coupling unit.

The probe cap 120 may include a housing 121 and a buffer 122. The housing 121 may form an external shape of the probe cap 120 and may protect the probe body 110 from a damage caused by an external force when being coupled with the probe body 110. The housing 121 may have various external shapes and may be generally formed corresponding to the external shape of the probe body 110. A material of the housing 121 may have strength for protecting the probe body 110 from the external force and not to be damaged from dropping impacts. Additionally, an inside of the housing 121 may have a property of reflecting ultrasound emitted from the probe body 110 while being coupled with the probe body 110 and performing a probe test. Accordingly, the inside of the housing 121 may have a material having an ultrasound reflection coefficient for allowing the probe test. Otherwise, a material layer having the ultrasound reflection coefficient for allowing the probe test may be additionally formed on an inner surface of the housing 121.

The buffer 122 is formed on the inside of the housing 121. The buffer 122 may be formed with a full area on the inner surface of the housing 121 to be in surface contact with the front end of the probe body 110 while the probe cap 120 is being coupled with the probe body 110.

While the housing 121 is primarily protecting the probe body 110 from external forces or impacts, the buffer 122 may secondarily protect the probe body 110 from the external forces or impacts. For this, the buffer 122 may be formed with a full thickness, material, and area for buttering impacts. For example, the buffer 122 may be formed of urethane rubber having acoustic impedance similar to the inside of the human body in order to be appropriate for an ultrasound probe test. In addition, medical silicone may be used.

Ultrasound is beyond an audible frequency not to be heard to a human ear. A range of the frequency thereof is more than 20 KHz. An ultrasound frequency range used for diagnosis is from about 2 MHz to about 15 MHz. In order to form a human diagnosis image using ultrasound, it is necessary to allow a short pulse within from about 2 to about 15 MHz to penetrate a human body. A signal returning from an interface of tissues is received and amplified and detected by a computer, thereby forming a two-dimensional image.

The acoustic impedance indicates impedance of a medium toward sound waves and has an effect on an amplitude and strength of a signal reflected by human tissue. The acoustic impedance is obtained by multiplying medium density p (kg/m³) by sound wave speed c (m/sec).

Z=p·c

A measure of the acoustic impedance is kg/m2/sec or rayls, in which 1 rayls corresponds to 1 kg/m2/sec. The acoustic impedance is proportional to the density.

When sound waves are incident to on a flat interface between two media having mutually different acoustic impedances, a part of them penetrates and another is reflected and returns. An ultrasound image is formed by the reflected and returning sound waves. An amount of reflection is determined depending on a difference between the densities of the two media, that is, a difference between acoustic impedances thereof. As the difference between the acoustic impedances becomes greater, a larger amount of reflection occurs.

When the acoustic impedances of the media are designated as Z1 and Z2, a reflection coefficient R is calculated as follows.

$$R = \frac{Z 2 - Z 1}{Z 2 + Z 1}$$

For example, since a reflection coefficient between lungs and the air is 99%, after that, ultrasound is hardly transferred. The reflection coefficient is regardless of a frequency of ultrasound but is determined depending on a difference between acoustic impedances.

As shown in FIG. 6, an echo of ultrasound may occur due to various physical phenomena such as reflection FIG. 6(a), scattering FIG. 6(b), and diffuse reflection FIG. 6(c).

The reflection may occur at a portion in which two media differing in acoustic impedance are in contact with each other with a broad area. The scattering may occur while being scattered from various angles when reflection of ultrasound is performed at a small object instead of a surface. In this case, strength of a returning echo is smaller than an echo returning from an interface between media. The diffuse reflection may occur when the interface between media is not even, which is similar to the reflection.

Accordingly, the housing 110 and the buffer 120 may be formed of materials with a great difference of acoustic impedance, in order to reflect a larger amount of ultrasound in the ultrasound probe test by using ultrasound reflection properties described above. Similarly, the housing 110 and the buffer 120 may have thicknesses, areas, forming positions, and shapes necessary to reflect a larger amount of ultrasound in the ultrasound probe test by using ultrasound reflection properties described above.

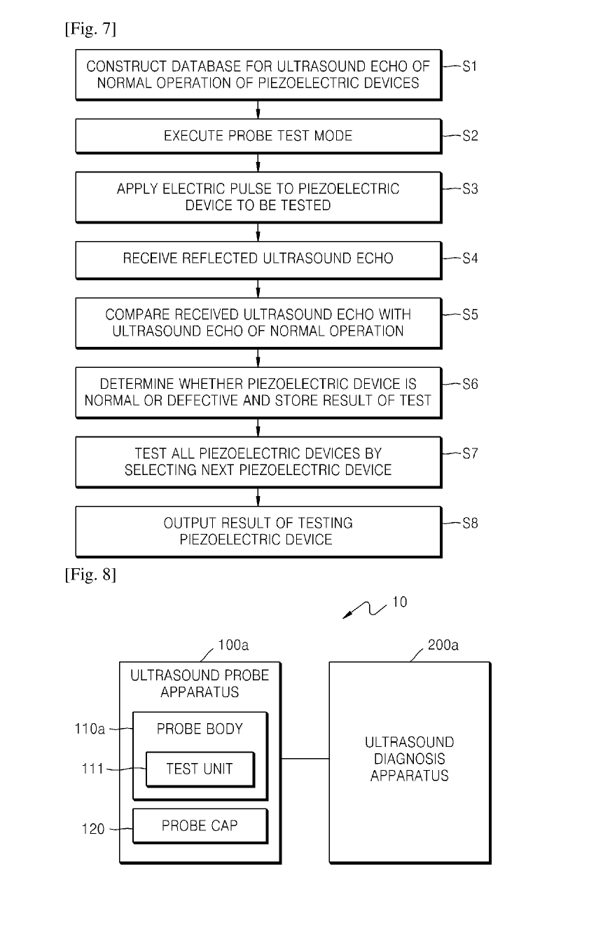

FIG. 7 is a flowchart illustrating the probe test method of the ultrasound diagnosis system 10 according to an embodiment of the present invention.

Referring to FIG. 7, the ultrasound diagnosis apparatus 200 constructs a database by forming ultrasound echo data corresponding to a normal operation in advance, in order to determine whether to operate with respect to an individual piezoelectric device included in the ultrasound probe apparatus 100 or not (S1).

The ultrasound diagnosis apparatus 200 executes a probe test mode according to an input of a user (S2). The probe body 110 generates ultrasound by applying an electric pulse to a piezoelectric device to be tested as the ultrasound diagnosis apparatus 200 executes the probe test mode (S3).

The probe body 110 receives an ultrasound echo reflected by the probe cap 120 (S4). The ultrasound echo received by the probe body 110 is transmitted to the ultrasound diagnosis apparatus 200.

The ultrasound diagnosis apparatus 200 compares the ultrasound echo received from the probe body 110 with the ultrasound echo data of the normal operation (S5).

The ultrasound diagnosis apparatus 200 determines whether the corresponding piezoelectric device is normal or not and stores a test result as normal or defective (S6).

The ultrasound diagnosis apparatus 200 selects a next piezoelectric device as an object of test and repetitively performs a test process described above with respect to all piezoelectric devices (S7).

The ultrasound diagnosis apparatus 200 outputs a final test result when testing the all piezoelectric devices is finally completed (S8).

FIG. 8 is a block view illustrating an ultrasound diagnosis system 10 according to another embodiment of the present invention.

Referring to FIG. 8, the ultrasound diagnosis system 10 may include an ultrasound probe apparatus 100a and an ultrasound diagnosis apparatus 200b.

The ultrasound probe apparatus 100a may include a probe body 110a and a probe cap 120. The probe cap 120 is coupled with a front end of the probe body 110a and protects the probe body 110a. In addition, the probe cap 120 may generate an ultrasound echo while testing piezoelectric devices of the probe body 110a.

The probe body 110a transmits an ultrasound signal to a subject in order to obtain an ultrasound image of the subject and receives and transmits the ultrasound signal reflected from the subject to the ultrasound diagnosis apparatus 200a. In addition, the probe body 110a includes a test unit 111. Accordingly, when the test unit 111 executes a probe test mode while the probe cap 120 is being coupled with the probe body 110a, the probe body 110a generates ultrasound for a probe test and receives an ultrasound echo reflected from the probe cap 120, thereby performing the probe test.

In addition, the probe body 110a may further include an ultrasound probe, a beam former, a data processor, a scan converter, and a display unit. The ultrasound probe transmits an ultrasound signal to an object and receives the ultrasound signal reflected from the object, that is, an ultrasound echo signal, thereby forming a reception signal. The ultrasound probe may include at least one transducer element operating to convert an ultrasound signal and an electric signal into each other. The beam former may analog/digital-convert the reception signal provided by the ultrasound probe, may delay a time of a digital signal considering a position and a focusing point of each transducer element, and forms ultrasound data, that is, RF data by summing up the time-delayed digital signals. The data processor performs various data processes with respect to ultrasound data, which are necessary for forming an ultrasound image. The scan converter scan-converts the processed ultrasound data to be displayed as an image. The display unit may display an operation state of the probe body 110a.

The ultrasound diagnosis apparatus 200a may include various devices having a wireless communication function and a display unit and operating application programs. For example, there may be a personal computer, a smartphone, a tablet type device, a pad type device, and a PDA.

The ultrasound diagnosis apparatus 200a may generate an ultrasound image of ultrasound data compensated with a time gain and controlled with brightness and contrast with respect to the ultrasound data received from the ultrasound probe apparatus 100a and may display the ultrasound image on the display unit.

The ultrasound diagnosis apparatus 200a may determine an ultrasound measurement depth according to an input of a user, may determine a parameter for adjusting the time gain based on the ultrasound measurement depth, and may determine degrees for adjusting brightness and contrast.

The ultrasound diagnosis apparatus 200a may receive the input of the user and may transmit the input to the ultrasound probe apparatus 100a. For example, the ultrasound diagnosis apparatus 200a may transmit the ultrasound measurement depth determined to the ultrasound probe apparatus 100a to control the ultrasound probe apparatus 100.

On the other hand, the ultrasound diagnosis apparatus 200a may receive the database for the ultrasound echo data, constructed in the ultrasound probe apparatus 100a and may manage the corresponding ultrasound probe apparatus 100a.

Also, the ultrasound diagnosis apparatus 200a may update management history data of the ultrasound probe apparatus 100a with the final test result received from the ultrasound probe apparatus 100a to manage a state of the ultrasound probe apparatus 100a. For example, the ultrasound diagnosis apparatus 200a may determine whether the ultrasound probe apparatus 100a normally operates or not or whether the ultrasound probe apparatus 100a is defective or not to be displayed to the user and may provide the user with a point in time of replacing the ultrasound probe apparatus 100a.

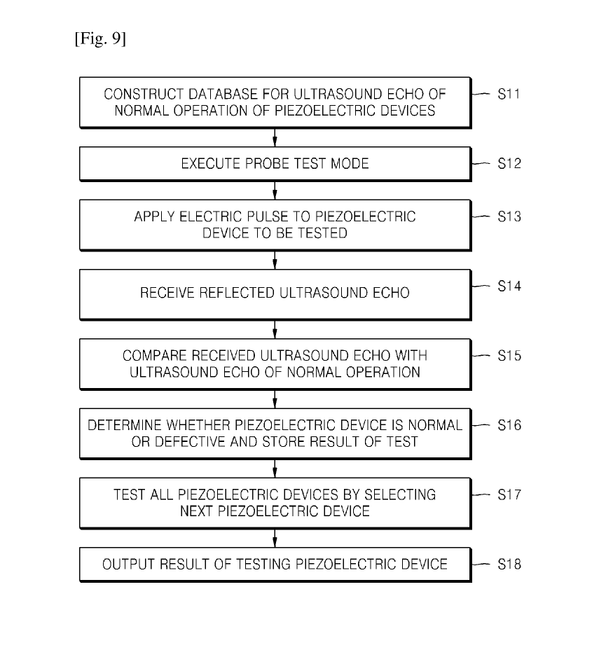

FIG. 9 is a flowchart illustrating a probe test method of the ultrasound diagnosis system 10 according to another embodiment of the present invention.

Referring to FIG. 9, the test unit 111 of the ultrasound probe apparatus 100a constructs a database by forming ultrasound echo data corresponding to a normal operation in advance, in order to determine whether to operate with respect to an individual piezoelectric device included in the ultrasound probe apparatus 100a or not (S11). The database for the ultrasound echo data, constructed in the ultrasound probe apparatus 100a, may be transmitted to the ultrasound diagnosis apparatus 200a and may be used in the ultrasound diagnosis apparatus 200a to manage the corresponding ultrasound probe apparatus 100a.

The test unit 111 of the ultrasound probe apparatus 100a executes a probe test mode according to an input of a user (S12). The probe body 110a generates ultrasound by applying an electric pulse to a piezoelectric device to be tested as the test unit 111 executes the probe test mode (S13).

The probe body 110a receives an ultrasound echo reflected by the probe cap 120 (S14).

The test unit 111 of the ultrasound probe apparatus 100a compares the ultrasound echo received from the probe body 111 with the ultrasound echo data of the normal operation (S15).

The test unit 111 of the ultrasound probe apparatus 100a determines whether the corresponding piezoelectric device is normal or not and stores a test result as normal or defective (S16).

The test unit 111 of the ultrasound probe apparatus 100a selects a next piezoelectric device as an object of test and repetitively performs a test process described above with respect to all piezoelectric devices (S17).

The test unit 111 of the ultrasound probe apparatus 100a outputs a final test result when testing the all piezoelectric devices is finally completed (S18).

According to a request of the user, the test unit 111 of the ultrasound probe apparatus 100a may transmit the final test result to the ultrasound diagnosis apparatus 200a. Also, the ultrasound diagnosis apparatus 200a may update management history data of the ultrasound probe apparatus 100a with the final test result received from the ultrasound probe apparatus 100a to manage a state of the ultrasound probe apparatus 100a.

While this invention has been particularly shown and described with reference to preferred embodiments thereof, it will be understood by those skilled in the art that various changes in form and details may be made therein without departing from the spirit and scope of the invention as defined by the appended claims.

1. An ultrasound diagnosis system comprising:
an ultrasound probe apparatus transmitting an ultrasound signal to obtain an ultrasound image of a subject, receiving the ultrasound signal reflected from the subject, emitting ultrasound to a probe cap to test a provided piezoelectric device module, and receiving an ultrasound echo obtained from the probe cap; and
an ultrasound diagnosis apparatus constructing a database for ultrasound echo data of a normal operation of the piezoelectric device module provided in the ultrasound probe apparatus, executing a probe test mode for testing an operation of the piezoelectric device module, receiving data of the ultrasound echo obtained from the probe cap from the ultrasound probe apparatus, comparing with the ultrasound echo data of the normal operation of the piezoelectric device module in the database, and displaying whether the piezoelectric device module is normal or defective as a test result.

2. The system of claim 1, wherein the ultrasound probe apparatus comprises:
a probe body transmitting the ultrasound signals to obtain the ultrasound image of the subject and to perform the probe test and receiving and transmitting the reflected ultrasound signals to the ultrasound diagnosis apparatus; and
a probe cap coupled with a front end of the probe body, protecting the probe body and generating the ultrasound echo while testing the piezoelectric device module of the probe body.

3. The system of claim 2, wherein the probe cap comprises:
a housing forming an external shape of the probe cap and protecting the probe body from a damage caused by an external force while being coupled with the probe body, an inside thereof formed of a material having an ultrasound reflection coefficient to allow the probe test; and
a buffer formed on an inner surface of the housing to be allowed to be in surface contact with the front end of the probe body while the probe cap is being coupled with the probe body and formed of a buffering material having an ultrasound reflection coefficient corresponding to acoustic impedance inside a human body to allow the probe test.

4. (canceled)

5. An ultrasound probe apparatus forming an ultrasound diagnosis system together with an ultrasound diagnosis apparatus, the ultrasound probe apparatus comprising:
a probe body transmitting an ultrasound signal to obtain an ultrasound image of a subject to the subject, receiving the ultrasound signal reflected from the subject, emitting ultrasound to test a provided piezoelectric device module, and receiving an ultrasound echo reflected therefrom; and
a probe cap coupled with a front end of the probe body, protecting the probe body and generating the ultrasound echo while testing the piezoelectric device module of the probe body

6. The apparatus of claim 5, wherein the probe cap comprises:
a housing forming an external shape of the probe cap and protecting the probe body from a damage caused by an external force while being coupled with the probe body, an inside thereof formed of a material having an ultrasound reflection coefficient to allow the probe test; and
a buffer formed on an inner surface of the housing to be allowed to be in surface contact with the front end of the probe body while the probe cap is being coupled with the probe body and formed of a buffering material having an ultrasound reflection coefficient corresponding to acoustic impedance inside a human body to allow the probe test.

7. (canceled)
